(19) United States
(12) Patent Application Publication
(10) Pub. No.: US 2005/0042682 A1
(43) Pub. Date: Feb. 24, 2005

(54) System and method for scoring peptide mass fingerprinting

(75) Inventors: Jacques Colinge, Neydens (FR); Jerome Magnin, Plan les Ouates (CH)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: GENEVA BIOINFORMATICS S.A., Geneva (CH)

(21) Appl. No.: 10/889,188

(22) Filed: Jul. 13, 2004

Related U.S. Application Data
(60) Provisional application No. 60/487,390, filed on Jul. 15, 2003.

(51) Int. Cl. G01N 33/53; G06F 19/00; G01N 33/48; G01N 33/50

(52) U.S. Cl. 435/7.1; 702/19

(57) ABSTRACT

The present disclosure relates to a system and method for scoring peptide mass fingerprinting. In one exemplary embodiment, a method for scoring peptide mass fingerprinting may comprise the steps of: providing a first list of peptide masses and a second list of peptide masses; defining a match between the first list of peptide masses and the second list of peptide masses based on one or more match components; calculating a first probability for observing the match based on a first hypothesis that the first list of peptide masses originates from a protein sample from which the second list of peptide masses originates; calculating a second probability for observing the match based on a second hypothesis that the first list of peptide masses does not originate from the protein sample from which the second list of peptide masses originates; and scoring the match between the first list of peptide masses and the second list of peptide masses based at least in part on a ratio between the first probability and the second probability.

### CROSS-REFERENCE TO RELATED APPLICATIONS

This patent application claims priority to U.S. provisional patent application No. 60/487,390, filed Jul. 15, 2003, entitled “Novel Scoring Scheme For High-Throughput Peptide Mass Fingerprinting,” which is hereby incorporated by reference in its entirety.

### FIELD OF THE DISCLOSURE

The present disclosure relates generally to protein and peptide analysis and, more particularly, to a system and method for scoring peptide mass fingerprinting.

### BACKGROUND OF THE DISCLOSURE

Mass Spectrometry (MS) combined with database searching has become the preferred method for identifying proteins in the context of proteomics projects. In a typical proteome project, a protein of interest may be digested into a mixture of peptides. A mass spectrum of the peptide mixture may provide a peptide mass fingerprint (PMF) with sufficient specificity to identify the original protein. Protein identification based on PMF is also known as “peptide mass fingerprinting.” It is particularly well adapted to high-throughput processes such as proteome-scale analysis of biological samples. This is typically the case when matrix assisted laser desorption/ionization time-of-flight (MALDI-TOF) technique is applied on samples comprising enzymatically digested protein(s). A typical peptide mass fingerprinting process may involve the following steps:
1. Purification and complexity reduction by protein separation techniques such as liquid-phase chromatography (LC) or gel electrophoresis separation (2D-PAGE).
2. Proteolytic treatment of protein fractions by an enzyme. A member of the trypsin family may be the usual choice, which offers excellent cleavage specificity and efficiency. During this phase, each protein present in the sample gives rise to a set of cleavage products (e.g., tryptic peptides in the case of trypsin).
3. Acquisition of mass spectrum, for every digested fraction, over a well-chosen mass range by a mass spectrometer. A signal detection algorithm (e.g., de-isotoping and charge-detection) is then applied to the raw spectrum to obtain a list of peptide masses potentially present in the analyzed sample. The mass list is often referred to as the experimental spectrum.
4. In silico comparison of the experimental set of peptide masses to theoretical ones generated from known protein sequences (e.g., those taken from a protein database such as SWISS-PROT) based on the known cleavage specificity of the enzyme used for digestion.

In practice, the comparison between experimental and theoretical spectra (or mass lists) typically results in a numerical value known as a score attributed to this comparison. In certain cases, several values are computed in addition to the score, such as a Z-score (normalized score), a p-value (the probability to obtain a larger or equal score by random chance), and the protein coverage, for example. Such values are typically the output of a scoring scheme, which are aimed at delivering an optimal compromise between sensitivity and selectivity while offering a sensible quantitative measure of the degree of coincidence between the experimental spectrum and the theoretical one. When available, additional quantities like the p-value often serve as decision-helpers by characterizing the degree of significance of the match between the theoretical and experimental spectra.

Interestingly, the amount of heuristics these existing scoring schemes rely upon varies noticeably from case to case. For example, the ProFound score (Zhang et al., 2000) is derived in a well-established and constraining Bayesian framework, on the basis of few ad hoc hypotheses. Heuristics are only present through an empirical factor that reacts to the presence of eventual “digestion patterns”. Another system such as MSA (Egelhofer et al., 2000), however, is highly heuristic both in the implemented procedure (re-calibration) and score formula. These two typical approaches are very different in nature, but both offer a level of performance making them subject of consideration by the MS community. In the detailed description that follows, embodiments of the present disclosure are compared with in-house implementations of these two well-established scoring schemes, which are hereafter referred to as “ProFound-like” and “MSA-like.”

### SUMMARY OF THE DISCLOSURE

According to the present disclosure, a system and method for scoring peptide mass fingerprinting is disclosed. In one particular exemplary embodiment, a method for scoring peptide mass fingerprinting may comprise the steps of: providing a first list of peptide masses and a second list of peptide masses; defining a match between the first list of peptide masses and the second list of peptide masses based on one or more match components; calculating a first probability for observing the match based on a first hypothesis that the first list of peptide masses originates from a protein sample from which the second list of peptide masses originates; calculating a second probability for observing the match based on a second hypothesis that the first list of peptide masses does not originate from the protein sample from which the second list of peptide masses originates; and scoring the match between the first list of peptide masses and the second list of peptide masses based at least in part on a ratio between the first probability and the second probability.

In accordance with another particular exemplary embodiment of the present disclosure, a computer readable medium having code for causing a processor to score peptide mass fingerprinting may comprise: code adapted to provide a first list of peptide masses and a second list of peptide masses; code adapted to define a match between the first list of peptide masses and the second list of peptide masses based on one or more match components; code adapted to calculate a first probability for observing the match based on a first hypothesis that the first list of peptide masses originates from a protein sample from which the second list of peptide masses originates; code adapted to calculate a second probability for observing the match based on a second hypothesis that the first list of peptide masses does not originate from the protein sample from which the second list of peptide masses originates; and code adapted to score the match between the first list of peptide masses and the second list of peptide masses based at least in part on a ratio between the first probability and the second probability.

In accordance with yet another particular exemplary embodiment of the present disclosure, a system for scoring peptide mass fingerprinting may comprise: means for providing a first list of peptide masses and a second list of peptide masses; means for defining a match between the first list of peptide masses and the second list of peptide masses based on one or more match components; means for calculating a first probability for observing the match based on a first hypothesis that the first list of peptide masses originates from a protein sample from which the second list of peptide masses originates; means for calculating a second probability for observing the match based on a second hypothesis that the first list of peptide masses does not originate from the protein sample from which the second list of peptide masses originates; and means for scoring the match between the first list of peptide masses and the second list of peptide masses based at least in part on a ratio between the first probability and the second probability.

In accordance with still another particular exemplary embodiment of the present disclosure, a protein-matching method for diagnosing diseases may comprise: providing a first list of peptide masses and a second list of peptide masses, wherein the first list of peptide masses is associated with at least one disease, and the second list of peptide masses is not associated with the at least one disease; defining a match between the first list of peptide masses and the second list of peptide masses based on one or more match components; calculating a first probability for observing the match based on a first hypothesis that the first list of peptide masses originates from a protein sample from which the second list of peptide masses originates; calculating a second probability for observing the match based on a second hypothesis that the first list of peptide masses does not originate from the protein sample from which the second list of peptide masses originates; scoring the match between the first list of peptide masses and the second list of peptide masses based at least in part on a ratio between the first probability and the second probability; and making diagnosis associated with the at least one disease based at least in part on the scored match.

The present disclosure will now be described in more detail with reference to exemplary embodiments thereof as shown in the appended drawings. While the present disclosure is described below with reference to preferred embodiments, it should be understood that the present disclosure is not limited thereto. Those of ordinary skill in the art having access to the teachings herein will recognize additional implementations, modifications, and embodiments, as well as other fields of use, which are within the scope of the present disclosure as disclosed and claimed herein, and with respect to which the present disclosure could be of significant utility.

### BRIEF DESCRIPTION OF THE DRAWINGS

In order to facilitate a fuller understanding of the present disclosure, reference is now made to the appended drawings. These drawings should not be construed as limiting the present disclosure, but are intended to be exemplary only.

FIG. 1 illustrates the significance of an amino acid composition bias factor in accordance with an embodiment of the present disclosure

FIG. 2 illustrates exemplary sequence coverage probability densities observed for a null-model and a training set in accordance with an embodiment of the present disclosure.

FIG. 3 illustrates a performance comparison between an exemplary OLAV-PMF implementation and two prior art scoring schemes in accordance with an embodiment of the present disclosure.

FIG. 4 is a block diagram illustrating an exemplary computer-based system for scoring peptide mass fingerprinting in accordance with an embodiment of the present disclosure.

### DETAILED DESCRIPTION OF THE DISCLOSURE

The disclosed system and method relate to a novel PMF scoring scheme named “OLAV-PMF.” The OLAV-PMF scoring scheme is based on a concept of probabilistic model of a match
M=M(Sₑₓₚ, Sₜₕ) (1)
between a list of experimental peptide masses, Sₑₓₚ, and a list of theoretical peptide masses, Sₜₕ, typically generated from a known protein sequence.

A score S may be constructed to provide an estimated ratio between two likelihood values, P(M|H₁) and P(M|H₀):
S=P(M|H₁)/P(M|H₀) (2)
where P(M|H₁) denotes a probability to effectively observe the match M, given that hypothesis H₁ is verified, and P(M|H₀) denotes a probability to effectively observe the match M, given that hypothesis H₀ is verified. H₁ is the alternative hypothesis, i.e., the hypothesis that Sₑₓₚ comes from a sample containing the protein from which Sₜₕ was generated. H₀ is the null hypothesis, i.e., the hypothesis that Sₜₕ comes from a protein sequence which is not in the sample.

Introduction of the score S may turn the protein identification problem into a classical hypothesis testing problem. The choice of the likelihood ratio as a test statistics is motivated by its demonstrated effectiveness. The probabilistic approach described herein differs noticeably from other existing scoring schemes for Mass Spectrometry. In a sense, the S score measures less an absolute degree of compatibility between the experimental and theoretical spectra than the level of significance or specificity relative to carefully modeled correct and random matches. The scoring scheme in accordance with the disclosed system and method therefore show some similarity to signal detection theory, which assesses sudden presence of a signal in a noisy background comprising protein mismatches.

Match Components

As used herein, the notion of match M is to be understood in a broad and flexible sense, in that it may include an arbitrary number l of components Sᵢ, i=1, . . . , l. Each of the components may address one particular aspect of the comparison between theoretical and experimental data. Explicitly, P(M|H₁) and P(M|H₀) may be re-written as:
P(M|H₁)=P(S₁, . . . ,Sₗ|H₁) (3a)
P(M|H₀)=P(S₁, . . . ,Sₗ|H₀) (3b)
In practice, a component Sᵢ of the match M may be typically represented by the statistical distribution of a well-defined quantity that may be considered a random variable. The quantity may be either continuous or discrete. Common examples of such quantities typical to PMF include, without limitation: peptide mass error, peptide amino acid composition, presence of a residue bearing a well-specified modification (e.g., methionine oxidation), number of missed cleavages, simultaneous match of a miscleaved peptide and one or more of its tryptic parts, and protein sequence coverage.

The match components may be distinguished according to a hierarchy, which is defined by decomposing a match into three parts:
1. Mass Match m: the match (at a prescribed mass tolerance) between a peak in the experimental peptide mass spectrum and a mass coming from the theoretical spectrum. The latter is the mass of one of the cleavage products of the candidate (or theoretical) protein. Several masses relating to the same cleavage product can be present in the experimental spectrum because of post-translational modifications (PTMs), thus yielding more than one mass match. According to some embodiments, the following potential modifications may be considered: cysteine carbo-amidomethy-lation (Cys_CAM) and oxidation of methionine, histidine and tryptophan.
2. Peptide Match Mₚ: the match of a set of Nₚ masses, Mₚ={m_{p,1}, . . . , m_{p,N}_{ₚ}}, to a specific cleavage product p.

3. Protein Match: the match of a set of P peptide mass sets, {Mp }, p=1 . . . P, to the peptide sequence of a specific protein.

According to some embodiments, it may be desirable to select, from all possible choices of Sᵢ, only those that offer a sufficiently strong discriminating power between hypotheses H₁ and H₀. For example, the match component Sᵢ may be selected by imposing a minimum ratio between probabilities P(Sᵢ|H₁) and P(Sᵢ|H₀). In another embodiment, several match component Sᵢ_{₁}, . . . ,Sᵢ_{_{N}} may be selected simultaneously by imposing a minimum ratio between probabilities P(Sᵢ_{₁}, . . . ,Sᵢ_{_{N}}|H₁) and P(Sᵢ_{₁}, . . . ,Sᵢ_{_{N}}H₀). In yet another embodiment, the match component Sᵢ may be selected by imposing a minimum relative entropy between the distributions of Sᵢ under the hypotheses H₁ and H₀. In a further embodiment, several match components Sᵢ_{₁}, . . . ,Sᵢ_{_{N}} may be selected simultaneously by imposing a minimum relative entropy between the distributions of Sᵢ_{₁}, . . . ,Sᵢ_{_{N}} under the hypotheses H₁ and H₀.

Among a set of l candidate quantities Sᵢ complying to the above criterion, a subset l′ of them may not be statistically independent. Therefore it may be desirable to apply a selection criterion to several of them (e.g., {S₁, . . . , S_{l′}}) simultaneously.

According to some embodiments, an ad hoc statistical independence assumption may be adopted, where P(S₁, . . . ,S_{l′}|H₁) and P(S₁, . . . ,S_{l′}|H₀) may be factorized with respect to the set of independent quantities. According to another embodiment, it may be assumed that all Sᵢ are independent. This approximation performs quite well as can be demonstrated a posteriori by monitoring the achieved performance. Those skilled in the art of machine learning would appreciate that naive Bayesian classifiers, built on the same independence assumption, frequently perform well.

Model Parameters and Training Sets

For effective implementation of the probabilistic approach, it may be desirable to have a training set of matches in the H₁ case, that is, a set of matches known to comprise correct protein identifications. The set should be large enough to allow the determination of alternative model parameters with satisfactory precision.

Correspondingly, a sufficiently large set of random matches in the H₀ case may be generated from a model of random sequences, which can be built as an order 3 Markov chain trained on SWISS-PROT human entries, for example.

The model parameters leamt in the H₁ and H₀ cases, together with the choice of the match components Sᵢ, are likely to depend on the sample preparation and actual settings of the MS instrument. It may not be possible to a priori provide a quantitative prediction of the impact of such dependence. Hence, changes in sample preparation or acquisition parameters may require a re-evaluation of model parameters from an updated training set.

Exemplary Implementation of OLAV-PMF

In one embodiment of the disclosed system, score S may be computed for each candidate protein sequence as:

$$S = {L}_{cov} {\prod }_{p = 1}^{P} \{ {〈 {L}_{c m} {L}_{comp} 〉}_{M ( p )} \} \qquad ( 4 )$$

where each Lᵢ may take the form of a likelihood ratio P(Sᵢ|H₁)/P(Sᵢ|H₀).

In Eq. (4), L_{cm} may be the likelihood ratio of a match component modeling the PTMs of a matched peptide. For every pair (modification Mᵢ, affected residue R(Mᵢ)), a probability P(Mᵢ, R(Mᵢ)|H₁) to observe the residue R(Mᵢ) in the modified state Mᵢ may be determined. Thus, L_{cm} may be built as the ratio of two products of binomial terms, which is, after simplification:

$$\begin{matrix}{L}_{c m} = {\prod }_{{M}_{i}} {( \frac{P ( {M}_{i} , R ( {M}_{i} ) \vert {H}_{1} )}{P ( {M}_{i} , R ( {M}_{i} ) \vert {H}_{0} )} )}^{{N}_{mod} ( R ( {M}_{i} ) )} \\ {( \frac{1 - P ( {M}_{i} , R ( {M}_{i} ) \vert {H}_{1} )}{1 - P ( {M}_{i} , R ( {M}_{i} ) \vert {H}_{0} )} )}^{{N}_{tot} ( R ( {M}_{i} ) ) - {N}_{mod} ( R ( {M}_{i} ) )}\end{matrix} \qquad ( 5 )$$

where Nₜₒₜ(R(Mᵢ)) is the total number of residues of type R(Mᵢ) found in the peptide, and N_{mod}(R(Mᵢ)) is the number of the residues actually observed in the modified state. The actual values taken by these probabilities are listed in Table 1.

TABLE 1
Probabilities entering the computation of component L_{cm} [Eq. (5)].

| Mᵢ | R(Mᵢ) | P(Mᵢ, R(Mᵢ) \| H₁) | P(Mᵢ, R(Mᵢ) \| H₀) |
|---|---|---|---|
| Cys_CAM | Cys | 0.89 | 0.5 |
| Oxidation | Met | 0.93 | 0.5 |
| Oxidation | His | 0.02 | 0.5 |
| Oxidation | Trp | 0.21 | 0.5 |

In Eq. (4), L_{comp} may be a likelihood ratio of a match component built around an amino acid composition bias factor affecting every matched peptide. The role of L_{comp} is to capture any effective preference of the overall identification process (including separation, sample preparation technique, ionization method, acquisition parameters, etc.) for certain amino acids that would translate into a statistically significant bias in the observed peptide composition. Practically, L_{comp} may be built as follows:

$${L}_{comp} = {\prod }_{r ∈ R} {( \frac{{v}_{r}^{obs}}{{v}_{r}^{av}} )}^{{N}_{tot} ( r )} \qquad ( 6 )$$

where the product is taken over the residues r whose observed frequency Vᵣ^{obs} has been determined to be significantly over- or under-represented with respect to its average value vᵣ^{av} in the reference population, and Nₜₒₜ(r) is the total number of residues of type r found in the peptide.

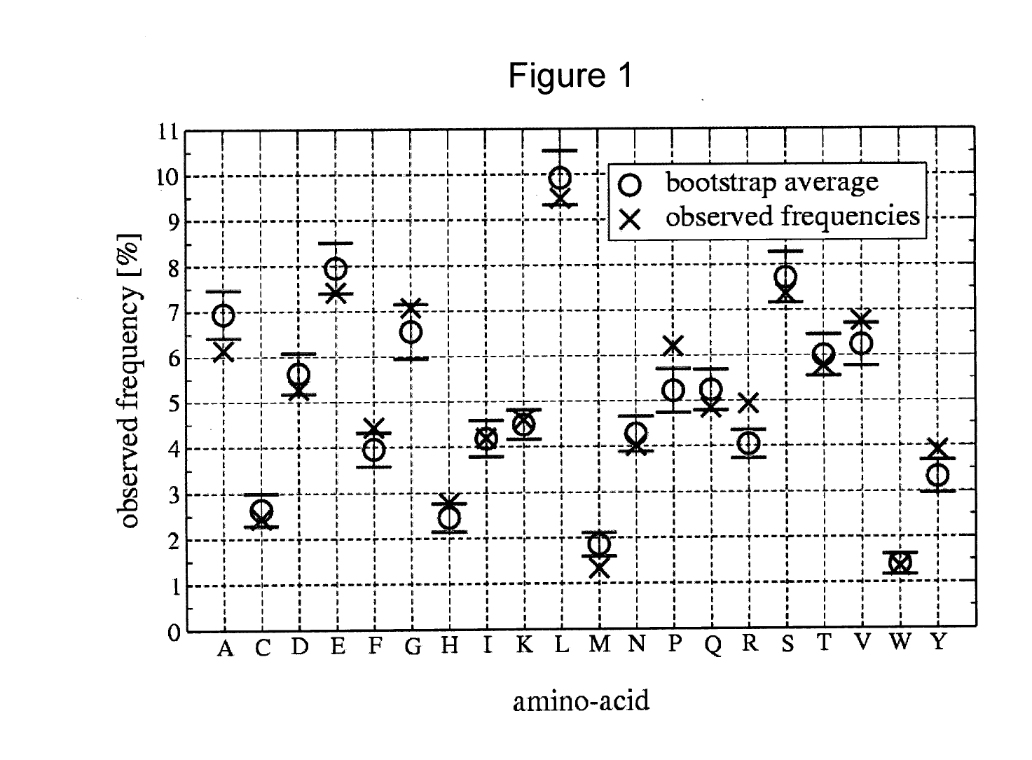

FIG. 1 illustrates the significance of an amino acid composition bias factor according to an embodiment of the present disclosure. In this particular embodiment, the limited size of the set of matched peptides observed in the training set (1450 peptides) makes it necessary to check that an apparent bias is statistically significant, i.e., it is not due to artifactual sampling effects. To achieve this validation, a bootstrap simulation is performed on a reference set comprising non-redundant population of all potentially observable peptides, that is, all peptides one can generate from the sequences of the proteins present in the training set. This reference set contains but does not equal the set of matched peptides, since not all peptides give rise to a signal in the mass spectrum. Then, N=2×10⁵ random selections are performed in the reference set, each selection yielding 1450 different peptides. For each selection, the amino acid frequencies are computed and recorded. Next, for each amino acid, the average and mean standard deviation of its related normal distribution of N frequencies are determined. In parallel, the amino acid frequencies are computed for the set of matched peptides. The two sets of 20 amino acid frequencies are shown in FIG. 1. The error bars mark the limits outside of which the observed frequencies (crosses) can be safely considered as significantly departing from the corresponding bootstrap-averaged frequency, thereby indicating a significant bias induced by the overall identification process.

Referring again to Eq. (4), the notation < . . . >_{M(p)} may be implemented in a variety of ways. For example, it may denote an average over the set M(p) of mass matches onto the peptide bearing index p. According to another embodiment of the present disclosure, the notation < . . . >_{M(p)} may denote the best mass match among the set M(p), i.e. the mass match having the highest likelihood ratio. According to yet another embodiment of the present disclosure, the notation < . . . >_{M(p)} may denote a weighted average, the individual weights being given. According to still another embodiment of the present disclosure, the notation < . . . >_{M(p)} may denote a weighted average, the individual weights being a function of the mass matches themselves.

In a further embodiment of the present disclosure, the individual weights may be a function of the mass matching precision, given assumed distributions of peptide mass errors in the cases H₁ and H₀. One exemplary choice may be a Gaussian distribution as experimental spectra generally represent the sum of many elementary measurements.

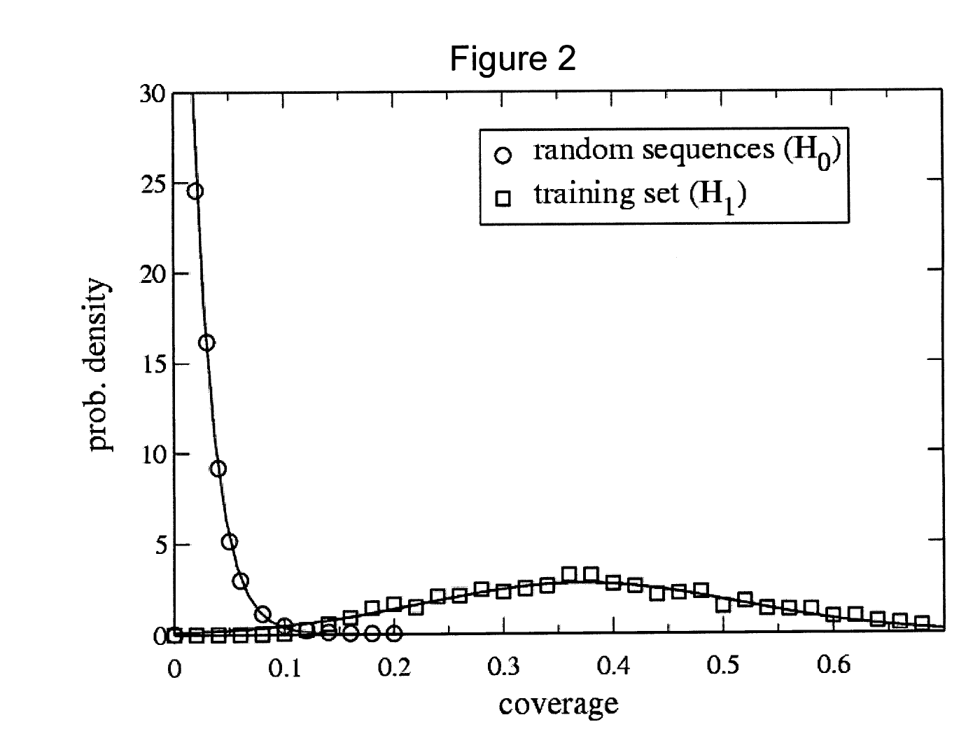

In Eq. (4), L_{cov} may be a sequence coverage rate likelihood ratio. It may be built around two continuous distributions defined over the real interval [0, 1], learnt from the two training sets in the H₁ and H₀ cases. FIG. 2 illustrates exemplary sequence coverage probability densities observed for the H₁ and H₀ cases according to an embodiment of the present disclosure. As shown in FIG. 1, the training set (H₁) sequence coverage can be fitted with a Gaussian distribution, while the null-model (H₀) can be fitted with an exponential distribution. These two distributions are sufficiently distinct from each other to furnish a significant contribution to S.

According to one embodiment of the present disclosure, before computing the actual score values for a database entry, the experimental mass list Sₑₓₚ may be pre-processed, with the help of Sₜₕ, based on the procedure described in (Egelhofer et al, 2002). This technique, applicable to MALDI-TOF spectra, may modify Sₑₓₚ in two ways: (i) it eliminates spurious, false positive mass matches, by drastically reducing the final mass tolerance used for match assignment; and (ii) it corrects systematic experimental deviations in mass values attributable to geometrical effects inside the mass spectrometer.

According to another embodiment of the present disclosure, an additional component L_{signal} may be introduced to provide discrimination (on the basis of the experimental peak signal properties) between masses corresponding to a successful identification and masses not corresponding to a peptide. Such a component can be based on a Fisher linear discriminant.

Detailed Example and Comparison to ProFound and MSA

To facilitate a better understanding of the OLAV-PMF scoring scheme, a detailed example is described below, where a comparison with the ProFound-like and MSA-like scoring schemes is also provided.

In this example, the training set associated with the alternative hypothesis H₁ comprises 4073 MALDI-TOF spectra. It represents a total of 63 different human plasmatic proteins. Note that this number is far beyond the minimal size necessary to obtain satisfactorily precise model parameters. For instance, it is found that a training set size of approximately 1000 different spectra does not result in any significant change in the parameter values. The H₁ training set is collected by submitting a considerably larger number of spectra to the MSA-like identification system, and by applying a conservative choice on the score value. For example, protein identifications with score S_{MSA-like}<99 are discarded. In addition, the H₁ training set retains only those remaining spectra that are matched to a protein whose presence among the samples have been established independently based on a LC-ESI-MS/MS ion trap analysis. This double screening ensures that the population of protein identifications in the H₁ training set is substantially dominated by correct protein identifications. 8477 independent spectra served as the H₁ training set.

The training set associated with the null hypothesis H₀ comprises 10⁷ pseudo-random protein sequences generated by the Makov chain model mentioned above. The sequence lengths distribution has been constrained to follow the distribution of sequence lengths of all SWISS-PROT entries in the ≧20 kDa molecular mass range, since this is a constant characteristic of the proteins present in the analyzed experimental fractions.

Each of the 8477 spectra (with known contents) in the H₁ training set is subjected to OLAV-PMF scoring. Every entry in the SWISS-PROT database has been attributed a score value based on Eqs (4)-(6), and has also been attributed ProFound-like and MSA-like scores (in-house implementation of the ProFound and MSA score schems is described below). For each of the three scoring schemes, only the 10 hits with highest scores are considered as potential true positives.

The same set of parameters is used with all three scoring schemes. Mass tolerance used in the recalibration process applied to Sₑₓₚ is 200 ppm. Mass tolerance used after recalibration, for scoring, is 50 ppm. Allowed variable modifications include Cys_CAM, oxidation of Met, His, and Trp. Maximum number of missed cleavages per peptide is 1.

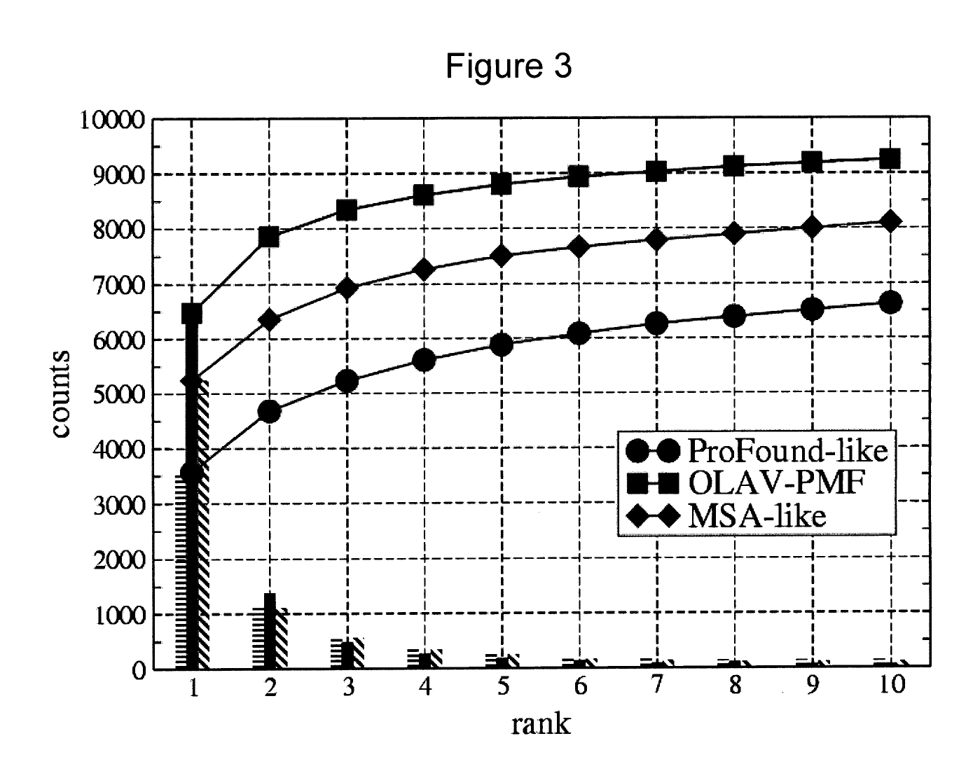

Based on the knowledge as to which database hit(s) are correct for every fraction, the rankings of the top-ten most hit database entries are summed up. In FIG. 3, the curves show the cumulated count of correct hits for the database entries ranked 1 through 10. The histogram bars show the individual numbers of correct hits achieved for each entry. For performance assessment purposes, results are shown for OLAV-PMF, as well as both ProFound-like and MSA-like scoring schemes.

Depending on the degree of manual validation one can afford in the identification results, different criteria may be considered as relevant to express in a quantitative manner the performance of a scoring scheme:
1. In a fully automated identification pipeline with no manual validation, a conservative and therefore sensible choice for such a criterion is the bare ability to correctly predict the database entry corresponding to the protein in the sample (“rank 1” occurrence count). FIG. 3 shows that the tested, relatively simple, three-components instance implementation of the proposed scoring scheme framework already performs significantly better (by 23.6%) than its nearest competitor MSA-like.
2. In an identification pipeline characterized by a time-limited human annotation step, a plausible scenario would be that automated procedures (a) skim the top n database hits off the hits list, and (b) submit them for manual annotation. In such a case, the relevant quantity is the cumulated count at n, plotted in FIG. 3. For n=5 (an arguably reasonable choice), OLAV-PMF outperforms MSA-like by 17.2%.

3. Finally, a somewhat more tolerant criterion would be the average rank. Such a quantity can be seen as an objective measure of the bare ability of a specific scorer to make the correct protein sequence “bubbles up” in the list of hits. It is, in this sense, the most “neutral” or context-independent indicator of the performance of a scoring scheme. Table 2 displays the values for the three candidates and also demonstrates the gain in performance obtained by the approach of the current disclosure.

TABLE 2
For each of the three scoring schemes considered:
average rank obtained by the test set.

| Scoring scheme | Average rank |
|---|---|
| OLAV-PMF | 1.73 |
| MSA-like | 2.02 |
| ProFound-like | 2.43 |

In-House Implementation of ProFound and MSA

For the comparison described above, the in-house implementation of ProFound is based on Eq. (3) of (Zhang et al., 2000), with the empirical term Fₚₐₜₜₑᵣₙ trivially set to 1, in the absence of any precise indication by the authors on how to implement this parameter. However, it is believed that this difference only marginally impacts the performance of the scoring scheme: manual inspection of many of the positive identifications has shown no real convincing evidence that overlapping and/or adjacent peptides form a real “signature” characterizing the population of experimentally matched peptides in a given spectrum.

The score value S_{ProFound-like} computed is then

$$\begin{matrix}{S}_{ProFound \text{-} like} = {( \sqrt{\frac{2}{π}} \frac{{m}_{max} - {m}_{min}}{N} )}^{r} \\ {\prod }_{i = 1}^{r} \{ {\sum }_{j = 1}^{{g}_{i}} exp ( - \frac{{( {m}_{i} - {m}_{i , j} )}^{2}}{2 {σ}_{i}^{2}} ) \}\end{matrix} \qquad ( 7 )$$

where [Mₘᵢₙ,Mₘₐₓ] denotes the acquisition mass range, N is the size of the theoretical mass list Sₜₕ, r is the number of mass hits, mᵢ is an experimental mass, mᵢⱼ is the theoretical masses matched by mᵢ at tolerance σᵢ, and gᵢ is the number of theoretical masses matching mᵢ.

An alternative version described in (Zhang et al., 2000b) and comprising an additional weighing by the local peptide masses density has also been implemented and tested. It is found to be comparable in performance to Eq. (7) above.

The in-house implementation of MSA follows exactly the description given in (Egelhofer et al., 2000 & 2002). S_{MSA-like} is computed as

$${S}_{MSA \text{-} like} = 100 - 500 F \frac{σ}{{n}^{2} γ} \qquad ( 8 )$$

where σ is the residual standard deviation of experimental mass errors after completion of the recalibration procedure, n is the number of matched masses, and γ is the sequence coverage. The parameter F—the only adjustable one of this scoring scheme—controls the sensitivity of the scorer. In the comparison above, an adapted value is used instead of the default one proposed in (Egelhofer et aL, 2002), to account for an average degree of quality slightly inferior in the experimental data than in the one reported by the reference article.
Computer-Based Implementation

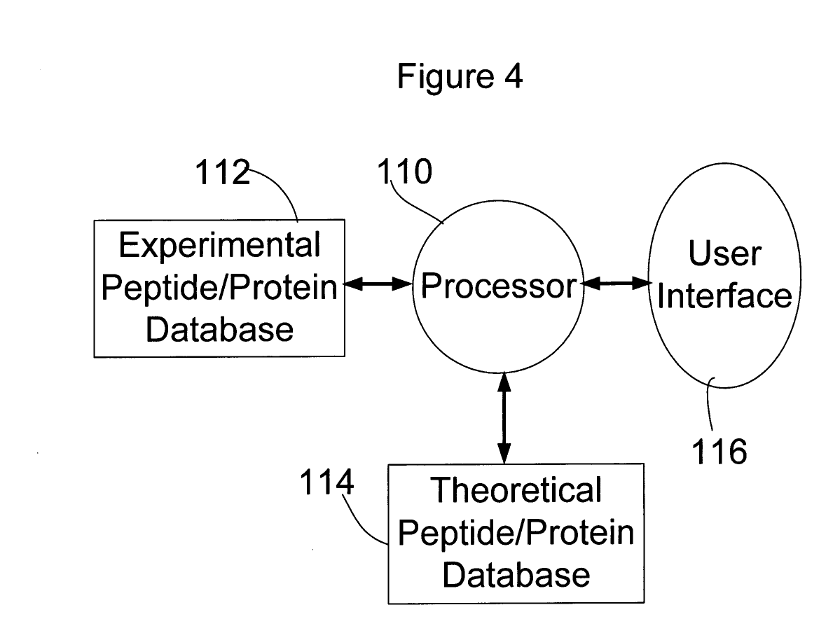

Referring to FIG. 4, there is shown a block diagram illustrating an exemplary computer-based system for scoring peptide mass fingerprinting in accordance with an embodiment of the present disclosure. The system may comprise Processor 110, Experimental Peptide/Protein Database 112, Theoretical Peptide/Protein Database 114 and User Interface 116. According to embodiments of the disclosure, the system may be implemented on computer(s) or a computer-based network. Processor 110 may be a central processing unit (CPU) or a computer capable of data manipulation, logic operation and mathematical calculation. According to an embodiment of the disclosure, Processor 110 may be a standard computer comprising at least an input device, an output device, a processor device, and a data storage device storing a module that is configured so that upon receiving a request to identify mass spectrometry data, it performs the steps listed in any one of the exemplary methods described above. Experimental Peptide/Protein Database 112 may be one or more databases containing experimental data associated with one or more peptides and/or proteins to be identified. Theoretical Peptide/Protein Database 114 may be one or more peptide/protein libraries or databases containing information associated with known peptides and/or proteins. According to an embodiment of the disclosure, databases 112 and 114 may be implemented with a single database or separated databases. User Interface 116 may be a graphical user interface (GUI) serving the purpose of obtaining inputs from and presenting results to a user of the system. According to embodiments of the disclosure, the User Interface module may be a display, such as a CRT (cathode ray tube), LCD (liquid crystal display) or touch-screen monitor, or a computer terminal, or a personal computer connected to Processor 110.

The computer-based system may be used in a wide range of applications where peptides and proteins are to be identified. The systems of the disclosure may be designed to permits the steps of: a) accessing a database of nucleic acid or amino acid sequences and/or mass spectra, e.g., experimental spectra; b) inputting an experimental mass spectrum or information derived therefrom, and interrogating said database to identify one or more candidate protein sequences or mass spectra that are related to or derived from the same protein as, the protein for which the experimental mass spectrum is provided; and c) outputting or displaying information concerning said candidate proteins. Each candidate protein can thereby be associated with a score as disclosed herein. For example, the system can output a list of peptides (using an identifier or some other description such as amino acid sequence) and associated match scores. The score may be an indication of the probability or likelihood that a candidate protein is or is not related or corresponding to the mass spectrum, and/or that a candidate protein is more likely to correspond to the experimental protein than another candidate protein.

It should be appreciated that the methods and systems of the disclosure can be used with a number of different apparati and mass spectrometry protocols. The scoring system or model of the disclosure may be readily adapted to the experimental environment of interest. For example, the stochastic model itself, e.g., the match components that are to be considered and their degree of dependency on other factors, can be adapted. Also, the parameters used in weighting the effect of different match components in the overall score may be adapted. At least two ways of learning the parameters and model to be used are possible. One is to provide a data set (e.g. experimental spectra) which has been manually verified and adjust the parameters and model to obtain an improved scoring accuracy. Another method is to provide a set of known protein standards and adjust the parameters and model to obtain improved scoring accuracy.

It should also be appreciated that the system and method for scoring peptide mass fingerprinting as described in the present disclosure may be implemented in a stand-alone manner or be combined with or embedded in other hardware or software applications. For example, other software programs may operate by taking the output or by feeding the input of the present disclosure. Such implementations are intended to fall within the scope of the present disclosure.

At this point it should be noted that the system and method in accordance with the present disclosure as described above typically involves the processing of input data and the generation of output data to some extent. This input data processing and output data generation may be implemented in hardware or software. For example, specific electronic components may be employed in a computer and communication network or similar or related circuitry for implementing the functions associated with scoring peptide mass fingerprinting in accordance with the present disclosure as described above. Alternatively, one or more processors operating in accordance with stored instructions may implement the functions associated with scoring peptide mass fingerprinting in accordance with the present disclosure as described above. If such is the case, it is within the scope of the present disclosure that such instructions may be stored on one or more processor readable carriers (e.g. , an optical disk or a magnetic disk), or transmitted to one or more processors via one or more signals (e.g., downloaded over an Internet connection).

According to an embodiment of the present disclosure, the scoring method may be applied to diagnose diseases. For example, a protein associated with one or more diseases may be associated with a “healthy protein”, i.e. one that is not associated with any diseases. The scoring method may be applied to identify the differences in concentration between the two protein in a control (healthy) patient and a diseased patient to calibrate the diagnostic tool. Further, the scoring method may be applied to measure the two proteins in a patient whose diagnosis is unknown, and compared to the reference levels to yield a diagnostic answer. Diagnosis about the one or more diseases may be based on the matching score and/or the differences identified.

The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings. Thus, such other embodiments and modifications are intended to fall within the scope of the following appended claims. Further, although the present disclosure has been described herein in the context of a particular implementation in a particular environment for a particular purpose, those of ordinary skill in the art will recognize that its usefulness is not limited thereto and that the present disclosure may be beneficially implemented in any number of environments for any number of purposes. Accordingly, the claims set forth below should be construed in view of the full breadth and spirit of the present disclosure as disclosed herein. Furthermore, several references have been cited in the present disclosure. Each of the cited references is incorporated herein by reference.

1. A method for scoring peptide mass fingerprinting, the method comprising:
providing a first list of peptide masses and a second list of peptide masses;
defining a match between the first list of peptide masses and the second list of peptide masses based on one or more match components;
calculating a first probability for observing the match based on a first hypothesis that the first list of peptide masses originates from a protein sample from which the second list of peptide masses originates;
calculating a second probability for observing the match based on a second hypothesis that the first list of peptide masses does not originate from the protein sample from which the second list of peptide masses originates; and
scoring the match between the first list of peptide masses and the second list of peptide masses based at least in part on a ratio between the first probability and the second probability.

2. The method according to claim 1, wherein:
the first list of peptide masses originates from an experimental protein; and
the second list of peptide masses originates from one or more known proteins.

3. The method according to claim 1, wherein the one or more match components comprise at least one characteristics selected from a group consisting of:
peptide mass error;
peptide amino acid composition;
presence of a residue bearing a specific modification;
number of missed cleavages;
simultaneous match of a miscleaved peptide and one or more of its tryptic parts;
protein sequence coverage; and
any observable or derivable peptide characteristics.

4. The method according to claim 1 further comprising determining probability distributions for the one or more match components.

5. The method according to claim 1, wherein each of the one or more match components is categorized as a mass match, a peptide match, or a protein match.

6. The method according to claim 1 further comprising selecting the one or more match components based on their discriminating power between the first hypothesis and the second hypothesis.

7. The method according to claim 1 further comprising making one or more ad hoc statistical independence assumptions associated with the one or more match components.

8. The method according to claim 1 further comprising identifying a protein associated with the first list of peptide masses based at least in part on the ratio between the first probability and the second probability.

9. The method according to claim 1 further comprising providing a first training set of protein matches based on the first hypothesis and a second training set of protein matches based on the second hypothesis.

10. The method according to claim 9 further comprising re-defining the match based on the first training set and the second training set.

11. A system for scoring peptide mass fingerprinting, the system comprising:
means for providing a first list of peptide masses and a second list of peptide masses;
means for defining a match between the first list of peptide masses and the second list of peptide masses based on one or more match components;
means for calculating a first probability for observing the match based on a first hypothesis that the first list of peptide masses originates from a protein sample from which the second list of peptide masses originates;
means for calculating a second probability for observing the match based on a second hypothesis that the first list of peptide masses does not originate from the protein sample from which the second list of peptide masses originates; and
means for scoring the match between the first list of peptide masses and the second list of peptide masses based at least in part on a ratio between the first probability and the second probability.

12. The system according to claim 11, wherein:
the first list of peptide masses originates from an experimental protein; and
the second list of peptide masses originates from one or more known proteins.

13. The system according to claim 11, wherein the one or more match components comprise at least one characteristics selected from a group consisting of:
peptide mass error;
peptide amino acid composition;
presence of a residue bearing a specific modification;
number of missed cleavages;
simultaneous match of a miscleaved peptide and one or more of its tryptic parts;
protein sequence coverage; and
any observable or derivable peptide characteristics.

14. The system according to claim 11 further comprising means for determining probability distributions for the one or more match components.

15. The system according to claim 11, wherein each of the one or more match components is categorized as a mass match, a peptide match, or a protein match.

16. The system according to claim 11 further comprising means for selecting the one or more match components based on their discriminating power between the first hypothesis and the second hypothesis.

17. The system according to claim 11 further comprising means for making one or more ad hoc statistical independence assumptions associated with the one or more match components.

18. The system according to claim 11 further comprising means for identifying a protein associated with the first list of peptide masses based at least in part on the ratio between the first probability and the second probability.

19. The system according to claim 11 further comprising means for providing a first training set of protein matches based on the first hypothesis and a second training set of protein matches based on the second hypothesis.

20. The method according to claim 9 further comprising re-defining the match based on the first training set and the second training set.

21. A computer readable medium having code for causing a processor to score peptide mass fingerprinting, the computer readable medium comprising:
code adapted to provide a first list of peptide masses and a second list of peptide masses;
code adapted to define a match between the first list of peptide masses and the second list of peptide masses based on one or more match components;
code adapted to calculate a first probability for observing the match based on a first hypothesis that the first list of peptide masses originates from a protein sample from which the second list of peptide masses originates;
code adapted to calculate a second probability for observing the match based on a second hypothesis that the first list of peptide masses does not originate from the protein sample from which the second list of peptide masses originates; and
code adapted to score the match between the first list of peptide masses and the second list of peptide masses based at least in part on a ratio between the first probability and the second probability.

22. A protein-matching method for diagnosing diseases, the method comprising:
providing a first list of peptide masses and a second list of peptide masses, wherein the first list of peptide masses is associated with at least one disease, and the second list of peptide masses is not associated with the at least one disease;
defining a match between the first list of peptide masses and the second list of peptide masses based on one or more match components;
calculating a first probability for observing the match based on a first hypothesis that the first list of peptide masses originates from a protein sample from which the second list of peptide masses originates;
calculating a second probability for observing the match based on a second hypothesis that the first list of peptide masses does not originate from the protein sample from which the second list of peptide masses originates;
scoring the match between the first list of peptide masses and the second list of peptide masses based at least in part on a ratio between the first probability and the second probability; and
making diagnosis associated with the at least one disease based at least in part on the scored match.
